(19) United States
(12) Patent Application Publication
(10) Pub. No.: US 2010/0093005 A1
(43) Pub. Date: Apr. 15, 2010

(54) IDENTIFYING DUCT CANCERS

(76) Inventors: Laurence J. Miller, Scottsdale, AZ (US); Gregory M. Hayes, Scottsdale, AZ (US)

Correspondence Address: FISH & RICHARDSON P.C., PO BOX 1022, MINNEAPOLIS, MN, 55440-1022, US

(21) Appl. No.: 12/514,491

(22) PCT Filed: Nov. 12, 2007
(86) PCT No.: PCT/US07/84444

§ 371 Date: Dec. 7, 2009

Related U.S. Application Data
(60) Provisional application No. 60/858,911, filed on Nov. 13, 2006.

(51) Int. Cl. G01N 33/574; C07K 16/18

(52) U.S. Cl. 435/7.92; 530/387.9; 530/391.3

(57) ABSTRACT

This document relates to methods and materials involved in identifying mammals that are likely to have duct cancer. For example, this document provides methods and materials related to using the presence of a variant form of the secretin receptor in a mammal's blood to identify the mammal (e.g., human) as being likely to have duct cancer.

### STATEMENT AS TO FEDERALLY SPONSORED RESEARCH

This invention was made with government support under Grant R01-DK46577 awarded by National Institutes of Health. The government has certain rights in the invention.

### BACKGROUND

1. Technical Field

This document relates to methods and materials involved in identifying mammals that are likely to have duct cancer. For example, this document provides methods and materials related to using the presence of a variant form of the secretin receptor in a mammal's blood to identify the mammal (e.g., a human) as being likely to have duct cancer.

2. Background Information

Pancreatic carcinoma and cholangiocarcinoma are highly aggressive malignancies. Chemotherapy and radiation therapy have not been curative for these forms of cancer, and the success of surgical resection correlates inversely with the size and invasiveness of the tumor. While small, early lesions can be cured surgically, the neoplasms have typically progressed to being locally invasive or even metastatic at the time of clinical presentation. Patients with these forms of cancer can, therefore, have a poor prognosis. In support of this, pancreatic carcinoma is the fourth most common cause of cancer death, although it is only the fourteenth most common cancer.

### SUMMARY

This document provides methods and materials related to identifying mammals (e.g., humans) as being likely to have duct cancer, e.g., pancreatic or bile duct cancer. Pancreatic cancer and bile duct cancer, or cholangiocarcinoma, are highly aggressive malignancies associated with poor prognosis. Early diagnosis of these cancers is difficult because the symptoms (e.g., loss of appetite and weight, and jaundice) are non-specific and varied. Most duct cancers are, therefore, advanced at the time of diagnosis. Tests that facilitate earlier diagnosis can improve the prognosis and survival of patients with duct cancer, especially given that surgical resection has a higher success rate when the tumors are small and not invasive.

This document is based, in part, on the discovery that splice variants of the secretin receptor are present in pancreatic carcinomas and cholangiocarcinomas, but not in corresponding non-cancerous tissues. A splice variant of the secretin receptor can include an early truncation that results in production of a secreted product having utility as a diagnostic and/or prognostic marker. A splice variant also can include a frameshift that produces a different amino acid sequence useful in the development of a specific recognition modality. Translation of a variant mRNA can result in production of a secreted, soluble 111-residue polypeptide, referred to herein as SecR(Δexon 3,4). This document is also based, in part, on the discovery that SecR(Δexon 3,4) polypeptides can be detected in blood from pancreatic cancer patients, but not in blood from age-matched controls. A SecR(Δexon 3,4) polypeptide can be used as an early marker for pancreatic and biliary ductular adenocarcinomas that can have profound clinical importance. For example, such a biomarker can be useful in population screening for duct cancer.

In one aspect, a method for identifying a mammal as being likely to have duct cancer is provided. The method comprises, or consists essentially of, (a) determining whether a mammal has a variant SecR polypeptide, and (b) classifying the mammal as being likely to have duct cancer if the mammal has the variant SecR polypeptide, where the variant SecR polypeptide lacks at least a portion of the amino acid sequence set forth in SEQ ID NO:1, lacks at least a portion of the amino acid sequence set forth in SEQ ID NO:1 and SEQ ID NO:2, or comprises an amino acid sequence at least 80 percent identical to the sequence set forth in SEQ ID NO:5. The mammal can be human. The duct cancer can be pancreatic or bile duct cancer. The method can comprise using blood, serum, or plasma to assess the presence or absence of the variant SecR polypeptide. The variant SecR polypeptide can lack at least a portion of the amino acid sequence set forth in SEQ ID NO:1. The variant SecR polypeptide can lack at least a portion of the amino acid sequence set forth in SEQ ID NO:1 and SEQ ID NO:2. The variant SecR polypeptide can comprise the amino acid sequence set forth in SEQ ID NO:5. The method can comprise using an ELISA or mass spectrometry. The method can have the ability to detect the presence of 200 pM of the variant SecR polypeptide. The method can have the ability to detect the presence of 100 pM of the variant SecR polypeptide.

In another aspect, an antibody preparation is provided. The antibody preparation comprises, or consists essentially of, an antibody or fragment thereof, where the antibody or fragment thereof can bind to a polypeptide consisting of the amino acid sequence set forth in SEQ ID NO:5. The antibody or fragment thereof can not bind to a polypeptide consisting of the amino acid sequence set forth in SEQ ID NO:13. The antibody or fragment thereof can bind to the polypeptide with an affinity greater than 10⁸ mol⁻¹ and can bind, if at all, to a polypeptide consisting of the amino acid sequence set forth in SEQ ID NO:13 with an affinity less than 10⁶ mol⁻¹. The antibody can be a monoclonal antibody. The antibody or fragment thereof can comprise a label selected from the group consisting of an enzyme, streptavidin, avidin, a fluorescent molecule, a luminescent molecule, a bioluminescent molecule, and a radioactive molecule.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIG. 1 contains images of agarose gels separating amplicons generated in nested RT-PCR reactions designed to amplify the portions of human secretin transcripts between exons 2 and 5 from pancreatic tumor cell lines (left), matched benign pancreatic tissue (n) and malignant pancreatic carcinomas (t; middle), and benign and malignant liver tissue (right). RT-PCR reactions for actin were used to normalize levels of expression. Data are representative of three independent experiments.

FIG. 2 contains the nucleotide sequence (SEQ ID NO:16) of human SecR(Δexon 3,4). The location of each exon-exon splice junction is indicated. A predicted amino acid sequence encoded by this transcript is indicated, where the first 21 residues (−21 to −1) represent the leader sequence (dashed line beneath) and residues 44 to 111 represent a polypeptide sequence resulting from the change in reading frame downstream of the exon 2/5 junction. The locations of the two antigenic polypeptides used for generating the monoclonal antibodies are underlined and labeled.

FIG. 3A is a graph plotting counts of radioiodinated forms of antigenic polypeptides that were precipitated with 5G1 or 8F4 monoclonal antibodies using protein A/G agarose beads. FIG. 3B is a Western blot analyzing lysates from cells transfected with the HA-tagged human SecR(Δexon 3,4) pCEP4 construct using 5G1 or 8F4 antibodies. As a positive control, Western blotting was also performed using an anti-HA antibody. FIG. 3C is a Western blot analyzing concentrated supernatants from MiaPaCa2 cells transfected with the HA-tagged human SecR(Δexon 3,4) pCEP4 construct or the pCEP4 vector only using the 5G1 and anti-HA (control) monoclonal antibodies. The data are representative of two independent experiments.

FIG. 4A is a standard curve plotting absorbance versus serum concentration of a human SecR(Δexon 3,4) polypeptide measured using a sandwich ELISA assay. FIG. 4B is a graph plotting absorbance values measured in patient and control sera using the ELISA assay. The gray background represents the limit in sensitivity of this assay (about 200 pM). Those values above 200 pM are plotted with means and S.E.M. Data are representative of three independent experiments.

FIG. 5 contains a nucleotide (SEQ ID NO:17) and corresponding amino acid sequence (SEQ ID NO:13) of a human secretin receptor polypeptide.

FIG. 6 is graph plotting the amount (pM) of human SecR(Δexon 3,4) polypeptide in serum samples obtained from healthy controls, pancreatitis patients, or pancreatic cancer patients. Those values below the limit of sensitivity of this assay are charted at that limit of 100 pM.

### DETAILED DESCRIPTION

This document provides methods and materials related to identifying a mammal (e.g., a human) as being likely to have duct cancer (e.g., pancreatic carcinoma or cholangiocarcinoma). For example, this document provides methods and materials for determining whether a sample (e.g., a blood sample) from a mammal contains a variant SecR polypeptide. As disclosed herein, if a mammal contains a variant SecR polypeptide, then the mammal can be classified as being likely to have duct cancer. Such a mammal can undergo further diagnostic studies and possible treatment.

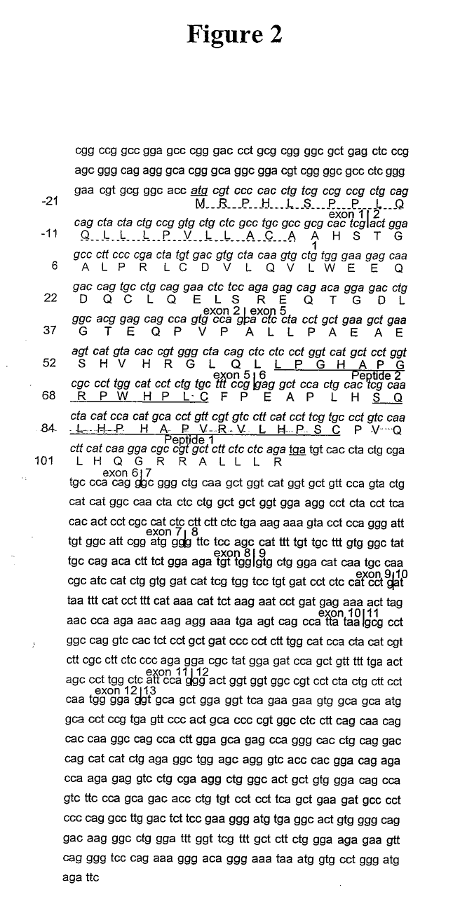

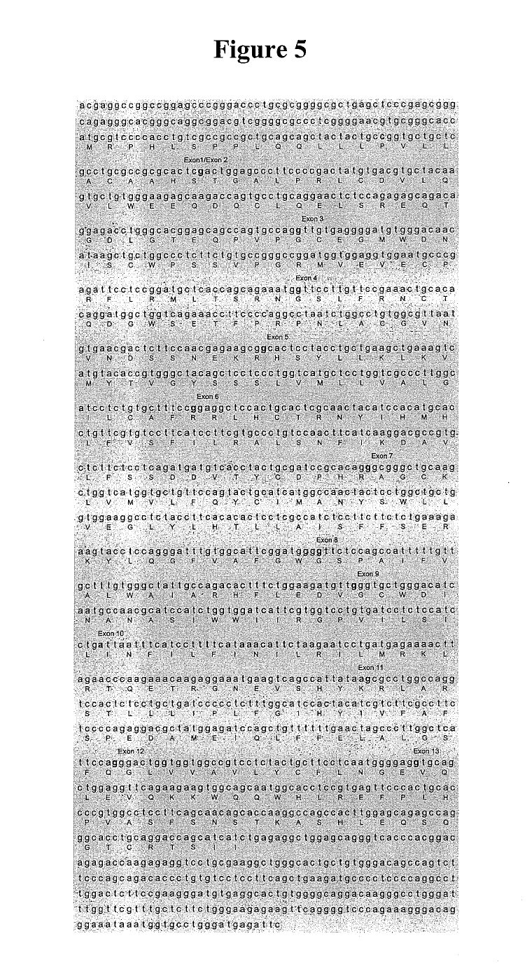

A variant SecR polypeptide can be any variant of a secretin receptor (e.g., a human secretin receptor; GenBank Accession No. NP_{—}002971). For example, a variant SecR polypeptide can be a SecR polypeptide that lacks the amino acid sequence encoded by exon 4, or a portion thereof. In some cases, a variant SecR polypeptide can lack the amino acid sequence corresponding to residues 80 to 114 (SEQ ID NO:1) of the amino acid sequence set forth in FIG. 5 (this numbering does not include the first 21 amino acid residues, which correspond to a leader sequence), or a portion thereof. In some cases, a variant SecR polypeptide can be a polypeptide having at least 80% sequence identity (e.g., at least 80, 85, 90, 95, or 99 percent identity) to a SecR polypeptide lacking the amino acid sequence encoded by exon 4, or a portion thereof. In some cases, a variant SecR polypeptide can be a SecR polypeptide that lacks the amino acid sequence encoded by each of exons 4 and 3, or a portion thereof. For example, a variant SecR polypeptide can lack the amino acid sequence corresponding to residues 80 to 114 (SEQ ID NO:1) and residues 44 to 79 (SEQ ID NO:2) of the amino acid sequence set forth in FIG. 5 (this numbering does not include the first 21 amino acid residues, which correspond to a leader sequence), or a portion thereof. In some cases, a variant SecR polypeptide can be a polypeptide having at least 80% sequence identity (e.g., at least 80, 85, 90, 95, or 99 percent identity) to a SecR polypeptide that lacks each of exons 4 and 3, or a portion thereof. In some cases, a variant SecR polypeptide can be a splice variant of a SecR polypeptide that lacks exons 3 and 4 and includes a frameshift and a truncation. The carboxy terminus of such a variant SecR polypeptide can have an amino acid sequence that is not present in wild-type SecR. For example, a variant SecR polypeptide can be a polypeptide comprising the amino acid sequence set forth in FIG. 2 (SEQ ID NO:3). In some cases, a variant SecR polypeptide can be a polypeptide having at least 80% sequence identity (e.g., at least 80, 85, 90, 95, or 99 percent identity) to the amino acid sequence set forth in SEQ ID NO:3. In some cases, a variant SecR polypeptide can be a polypeptide comprising residues 1-111 of the amino acid sequence set forth in FIG. 2 (SecR(Δexon 3,4); SEQ ID NO:4). In some cases, a variant SecR polypeptide can be a polypeptide having at least 80% sequence identity (e.g., at least 80, 85, 90, 95, or 99 percent identity) to the amino acid sequence set forth in SEQ ID NO:4. In some cases, a variant SecR polypeptide can comprise an amino acid sequence corresponding to residues 44 to 111 of the amino acid sequence set forth in FIG. 2 (SEQ ID NO:5). In some cases, a variant SecR polypeptide can comprise an amino acid sequence having at least 80% sequence identity (e.g., at least 80, 85, 90, 95, or 99 percent identity) to SEQ ID NO:5. A variant SecR polypeptide can comprise a leader sequence, such as a leader sequence corresponding to residues −21 to −1 of the amino acid sequence set forth in FIG. 2 (SEQ ID NO:6).

The percent identity between two nucleic acid sequences or two amino acid sequences is determined as follows. First, two nucleic acid sequences or amino acid sequences are compared using the BLAST 2 Sequences (Bl2seq) program from the stand-alone version of BLASTZ containing BLASTN version 2.0.14 and BLASTP version 2.0.14. This stand-alone version of BLASTZ can be obtained from Fish & Richardson's web site (World Wide Web at “fr” dot “com” slash “blast”), the U.S. government's National Center for Biotechnology Information web site (World Wide Web at “ncbi” dot “nlm” dot “nih” dot “gov”), or the State University of New York at Old Westbury Library (QH 497.m6714). Instructions explaining how to use the Bl2seq program can be found in the readme file accompanying BLASTZ. Bl2seq performs a comparison between two sequences using either the BLASTN or BLASTP algorithm. BLASTN is used to compare nucleic acid sequences, while BLASTP is used to compare amino acid sequences. To compare two nucleic acid sequences, the options are set as follows: -i is set to a file containing the first nucleic acid sequence to be compared (e.g., C:\seq1.txt); -j is set to a file containing the second nucleic acid sequence to be compared (e.g., C:\seq2.txt); -p is set to blastn; -o is set to any desired file name (e.g., C:\output.txt); -q is set to −1; -r is set to 2; and all other options are left at their default setting. For example, the following command can be used to generate an output file containing a comparison between two sequences: C:\B12seq -i c:\seq1.txt -j c:\seq2.txt -p blastn -o c:\output.txt -q −1 -r 2. To compare two amino acid sequences, the options of Bl2seq are set as follows: -i is set to a file containing the first amino acid sequence to be compared (e.g., C:\seq1.txt); -j is set to a file containing the second amino acid sequence to be compared (e.g., C:\seq2.txt); -p is set to blastp; -o is set to any desired file name (e.g., C:\output.txt); and all other options are left at their default setting. For example, the following command can be used to generate an output file containing a comparison between two amino acid sequences: C:\B12seq -i c:\seq1.txt -j c:\seq2.txt -p blastp -o c:\output.txt. If the two compared sequences share homology, then the designated output file will present those regions of homology as aligned sequences. If the two compared sequences do not share homology, then the designated output file will not present aligned sequences. Once aligned, the number of matches is determined by counting the number of positions where an identical nucleotide or amino acid residue is presented in both sequences.

The percent identity is determined by dividing the number of matches by the length of the sequence set forth in an identified sequence (e.g., SEQ ID NO:5) followed by multiplying the resulting value by 100. For example, if a sequence is compared to a sequence set forth in a sequence identifier with a length of 100 and the number of matches is 90, then the sequence has a percent identity of 90 (i.e., 90÷100*100=90) to the sequence set forth in that sequence identifier.

It is noted that the percent identity value is rounded to the nearest tenth. For example, 78.11, 78.12, 78.13, and 78.14 are rounded down to 78.1, while 78.15, 78.16, 78.17, 78.18, and 78.19 are rounded up to 78.2. It is also noted that the length value will always be an integer.

Any appropriate method can be used to determine whether a sample from a mammal contains a variant SecR polypeptide. For example, chromatography, mass spectrometry, or a surface plasmon resonance biosensor can be used to determine whether a sample contains a polypeptide comprising the amino acid sequence set forth in SEQ ID NO:5. In some cases, antibodies directed against a variant SecR polypeptide can be used (e.g., in an ELISA assay) to determine whether a sample contains a variant SecR polypeptide. Such antibodies can be labeled for detection. For example, an anti-SecR(Δexon 3,4) polypeptide antibody can be labeled with a radioactive molecule, a fluorescent molecule, or a bioluminescent molecule. In some cases, a labeled antibody can be used to detect an unlabeled antibody bound to a variant SecR polypeptide. In some cases, an antibody can be used to isolate a variant SecR polypeptide from a sample to increase the sensitivity of detection of the variant SecR polypeptide by any appropriate method, such as a method described herein.

An antibody can be, without limitation, a polyclonal, monoclonal, human, humanized, chimeric, or single-chain antibody, or an antibody fragment having binding activity, such as a Fab fragment, F(ab′) fragment, Fd fragment, fragment produced by a

Fab expression library, fragment comprising a VL or VH domain, or epitope binding fragment of any of the above. An antibody can be of any type, (e.g., IgG, IgM, IgD, IgA or IgY), class (e.g., IgG1, IgG4, or IgA2), or subclass. In addition, an antibody can be from any animal including birds and mammals. For example, an antibody can be a human, rabbit, sheep, or goat antibody. An antibody can be naturally occurring, recombinant, or synthetic. Antibodies can be generated and purified using any suitable methods known in the art. For example, monoclonal antibodies can be prepared using hybridoma, recombinant, or phage display technology, or a combination of such techniques. In some cases, antibody fragments can be produced synthetically or recombinantly from a gene encoding the partial antibody sequence. An antibody directed against a variant SecR polypeptide can bind to the variant SecR polypeptide at an affinity of at least 10⁴ mol⁻(e.g., at least 10⁵, 10⁶, 10⁷, 10⁸, 10⁹, 10¹⁰, 10¹¹ or 10¹² mol⁻¹).

The presence of a variant SecR polypeptide in a sample also can be determined by measuring the level of an RNA that encodes a variant SecR polypeptide. Any appropriate method can be used to measure the level of an RNA encoding a variant SecR polypeptide including, without limitation, PCR-based methods. For example, quantitative PCR can be used with fluorescent beacons or dyes and oligonucleotide primers designed to amplify nucleic acid (e.g., RNA) encoding a polypeptide comprising the amino acid sequence set forth in SEQ ID NO:5.

Any appropriate type of sample can be evaluated for the presence of a variant SecR polypeptide including, without limitation, a blood sample. In addition, any appropriate method can be used to obtain a sample. For example, a blood sample can be obtained by peripheral venipuncture. In some cases, a tissue sample can be obtained from a tissue biopsy (e.g., a needle biopsy). A sample can be manipulated prior to being evaluated for the presence of a variant SecR polypeptide. For example, a blood sample can be collected in the presence of an anticoagulant, such as heparin, and centrifuged to remove cellular elements. Highly abundant proteins, such as albumin and immunoglobulin G, can be removed. Blood, plasma, or serum samples also can be stored (e.g., frozen), diluted, or concentrated prior to analysis. In addition, nucleic acids and/or polypeptides can be extracted from a sample, purified, and evaluated for the presence of a variant SecR polypeptide.

The methods and materials provided herein for identifying mammals as being likely to have duct cancer can be used in combination with other methods and materials for identifying duct cancer. Such other methods and materials include, without limitation, imaging studies, such as ultrasound or computed tomography (CT), abdominal palpation, and review of the patient history. A mammal can be evaluated regularly for the likelihood of having duct cancer. For example, a mammal can be evaluated once a year for as long as the mammal is alive. In some cases, humans can be evaluated once every year beginning at age 35. Mammals that are susceptible to develop duct cancer can be screened more frequently, and screening can be started at an earlier age. For example, mammals having a genetic predisposition to develop cancer or a family history of cancer can be assessed more frequently. An evaluation of a mammal for the presence of a variant SecR polypeptide can be used to supplement other screening tests, such as a fasting serum glucose that is often elevated in pancreatic carcinoma.

Patients who might be at increased risk for developing pancreatic duct cancers include those with a positive family history of pancreatic cancer, smokers, new-onset diabetics, and those with chronic pancreatitis, hereditary pancreatitis, atypical mole-melanoma syndrome, BRCA2 defects in hereditary breast-ovarian cancer syndrome, and Peutz-Jeghers syndrome. Each of these may be preferred candidates for a screening test, such as a screening test to evaluate the patient for the presence of a variant SecR polypeptide as described herein. Further, patients presenting with unexplained weight loss, abdominal pain, jaundice, or wasting could benefit from a screening test for these cancers.

In some cases, a mammal (e.g., a human) presenting with unexplained weight loss, abdominal pain, jaundice, or wasting can be evaluated for pancreatitis using diagnostic techniques for pancreatitis. If the diagnostic techniques for pancreatitis (e.g., techniques such as an evaluation for a history of alcohol-related abdominal pain or elevated amylase and lipase levels) reveal that the mammal does not appear to have pancreatitis, then the mammal can be evaluated for the presence of a variant SecR polypeptide as described herein. The presence of a variant SecR polypeptide can indicate that the mammal is likely to have duct cancer. In such cases, further diagnostic techniques for duct cancer can be performed such as invasive imaging or tissue biopsy techniques to confirm the presence or absence of duct cancer.

Once duct cancer has been identified in a mammal, the mammal can continue to be monitored for the presence of a variant SecR polypeptide. For example, a mammal can be evaluated for the presence of a polypeptide comprising the amino acid sequence set forth in SEQ ID NO:5 during and after treatment for a duct cancer, e.g., to assess the efficacy of the therapy. For example, if it is determined that a sample (e.g., blood) obtained from a mammal following treatment does not contain a polypeptide comprising the amino acid sequence set forth in SEQ ID NO:5, whereas a comparable sample (e.g., blood) obtained from the mammal prior to treatment does contain a polypeptide comprising the amino acid sequence set forth in SEQ ID NO:5, then the treatment can be classified as being effective. In addition, if a sample obtained from a mammal at some time point following successful treatment is once again found to contain a variant SecR polypeptide, then the mammal can be classified as having recurring disease. In some cases, a mammal can be classified as having progression of duct cancer if it is determined that the amount of a variant SecR polypeptide in the mammal has increased over time.

This document also provides methods and materials to assist medical or research professionals in determining whether or not a mammal is likely to have duct cancer. Medical professionals can be, for example, doctors, nurses, medical laboratory technologists, and pharmacists. Research professionals can be, for example, principle investigators, research technicians, postdoctoral trainees, and graduate students. A professional can be assisted by (1) determining whether or not a sample contains a variant SecR polypeptide, and (2) communicating information about the presence, absence, or amount of variant SecR polypeptide to that professional.

Any method can be used to communicate information to another person (e.g., a professional). For example, information can be given directly or indirectly to a professional. In addition, any type of communication can be used to communicate the information. For example, mail, e-mail, telephone, and face-to-face interactions can be used. The information also can be communicated to a professional by making that information electronically available to the professional. For example, the information can be communicated to a professional by placing the information on a computer database such that the professional can access the information. In addition, the information can be communicated to a hospital, clinic, or research facility serving as an agent for the professional.

This document also provides kits for detecting variant SecR polypeptides. For example, this document provides kits containing antibodies and reagents for detecting variant SecR polypeptides. Such kits also can include reagents and supplies for collecting and processing samples, such as blood samples, for evaluation. In addition, such kits can include a standard for use as a control, such as a polypeptide standard, and instructions for detecting variant SecR polypeptides in mammals.

The invention will be further described in the following examples, which do not limit the scope of the invention described in the claims.

### Examples

### Example 1

### Identification of mRNA Encoding Secretin Receptor Variants in Pancreatic Carcinoma and Cholangiocarcinoma

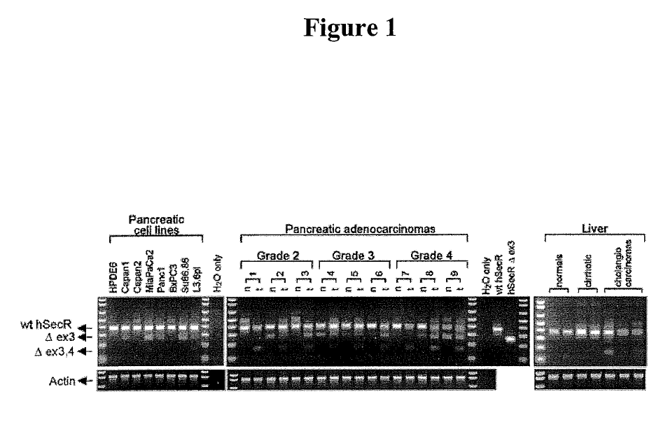

Molecular analysis of macro-dissected human pancreatic ductular adenocarcinomas demonstrated the existence of multiple forms of the secretin receptor (SecR) that were not present in normal pancreatic tissues (Korner et al., Am J Pathol., 167:959-968 (2005); Ding et al., Cancer Res., 62:5223-5229 (2002)). Similar mRNA splice variants were observed in cholangiocarcinoma samples, but not in benign or cirrhotic liver specimens. To characterize the secretin receptor variants observed in human pancreatic ductular adenocarcinomas and cholangiocarcinomas, nested RT-PCR reactions were performed using cDNAs produced from histologically well-characterized and graded tumor specimens (FIG. 1). Matched benign specimens from each pancreatic carcinoma patient were investigated in a similar manner using primers corresponding to exons 2 and 5 of the human SecR gene.

RNA was isolated from Trizol extracts of macroscopically-dissected human pancreatic and hepatic tissues. The tissues were ground in liquid nitrogen and resuspended in Trizol at a concentration of about 10 mg per mL Trizol. Total RNA was purified from 100 μL aliquots of each Trizol suspension using the Qiagen RNeasy Kit (Qiagen, Valencia, Calif.) and treated with DNase I (Invitrogen, Carlsbad, Calif.) to remove any genomic DNA. The integrity and concentration of the RNA were established by gel electrophoresis and spectrophotometry. Aliquots of 0.5 μg total RNA were used to produce cDNA with Promega's First Strand cDNA synthesis kit (Madison, Wis.).

Standard PCR reactions were performed using 25 ng of cDNA, Platinum Taq polymerase, dNTPs, and primers according to the manufacturer's protocol (Invitrogen). Nested RT-PCR amplification of human secretin receptor transcripts was performed using primers specific for the 5′ and 3′ regions of the cDNA for the first reaction (5′-GCAGCAGCTACTACTGCCGGTGC-3′ (SEQ ID NO:7) and 5′-AGCCTTCGCAGGA CCTCTCTTGG-3′ (SEQ ID NO:8), respectively). One microliter of the first PCR reaction was used as template for the nested reaction using primers specific for exons 2 and 5 of the secretin gene (5′-AGAGCAAGACCAGTGCCTGCAGG-3′ (SEQ ID NO:9) and 5′-AGAGGATGCCAAGGGCGACCAG-3′ (SEQ ID NO:10), respectively). Actin control reactions were performed using the primers 5′-CCAGCTCACCATGGAT GATGATATCG-3′ (SEQ ID NO:11) and 5′-GGAGTTGAAGGTAGTTTCGTG GATGC-3′ (SEQ ID NO:12). Amplification products were resolved on 2% agarose gels and representative bands were excised and sequenced to confirm their identities.

Sequencing of representative amplification products indicated the presence of a secretin receptor isoform missing exon 3, which is described elsewhere (Ding et al., Cancer Res., 62:5223-5229 (2002); Ding et al., Gastroenterology, 122:500-511 (2002)). In addition, an isoform was discovered in which both exons 3 and 4 had been excluded from the mRNA during processing. Exon 2 was inappropriately joined to exon 5 in this previously unidentified transcript, resulting in a new reading frame that extended from the junction between exons 2 and 5 and terminated in exon 6 (FIG. 2; SEQ ID NO:5). The slice variant is predicted to encode a 132 amino acid polypeptide (SEQ ID NO:3), with the first 21 residues representing a leader sequence (SEQ ID NO:6). The latter is cleaved to produce the mature secretin receptor (Dong et al., J Biol Chem., 274:19161-19167 (1999)). The mature polypeptide is predicted to comprise 111 residues (SEQ ID NO:4) with a molecular mass of about 14 kDa. This includes the first 43 residues that it shares with the amino terminus of the secretin receptor, and a novel reading frame that results in a 68 amino acid sequence (SEQ ID NO:5) that is unique in the transcriptome/proteome. The human SecR(Δexon 3,4) variant was detected in each of nine pancreatic tumor specimens, but not in surrounding normal pancreatic tissues. Similarly, the variant was detected in three cholangiocarcinoma specimens studied, but not in healthy or cirrhotic liver tissue.

RT-PCR reactions similar to those described above were also performed using cDNAs from a panel of pancreatic cancer cell lines and the near-normal human pancreatic ductular epithelial cell line, HPDE6 (Ouyang et al., Am J Pathol., 157:1623-1631 (2000); FIG. 1). The pancreatic ductal adenocarcinoma cell lines, Capan-1, Capan-2, MiaPaCa-2, Pancl, BxPC3, and Su86.86, were obtained from the American Type Culture Collection (ATCC, Manassas, Va.) and cultured according to ATCC specifications. The pancreatic cancer cell line, L3.6pl, was cultured in MEM medium supplemented with 10% fetal bovine serum, nonessential amino acids, sodium pyruvate, L-glutamine, and vitamin solution (Invitrogen, Carlsbad, Calif.). The near-normal human pancreatic ductular cell line, HPDE6, was grown in Keratinocyte-SFM medium supplemented with bovine pituitary extract and epidermal growth factor (Invitrogen), as described elsewhere (Furukawa et al., Am J Pathol., 148:1763-1770 (1996); Ouyang et al., Am J Pathol., 157:1623-1631 (2000)). The cells were allowed to grow to a maximum of about 80% confluency before being trypsinized and harvested, washed once in phosphate-buffered saline (PBS), and resuspended at 5×10⁷ cells per mL of Trizol (Invitrogen) for extraction of RNA.

Expression of mRNA encoding wild type human secretin receptor and the SecR(Δexon3) variant was detected in the majority of cell lines in vitro. However, the SecR(Δexon 3,4) variant was only detected at a low level in the L3.6pl cell line, and to a lesser extent in the MiaPaCa2 cell line. These results indicate that the mRNA encoding the SecR(Δexon 3,4) variant is not prominent in commonly studied pancreatic cancer cell line, but is clearly present in primary tumors of both pancreas and bile duct.

### Example 2

### Generation of Anti-Human SecR(ΔExon 3,4) Monoclonal Antibodies

To evaluate the utility of the SecR(Δexon 3,4) variant as a cancer biomarker, mouse monoclonal antibodies (mAbs) were raised against KLH-conjugated peptides representing two antigenic determinants unique to this secretin receptor variant (FIG. 2). Antigenicity was determined as described elsewhere (Kolaskar and Tongaonkar, FEBS Lett., 276:172-174 (1990)), via the EMBOSS explorer site. Two potentially unique antigenic determinants were identified via in silico examination of the amino acid sequence encoded by the 111-residue human SecR(Δexon 3,4) splice variant. The first 43 residues of this polypeptide were identical to the first 43 residues of the secretin receptor amino terminus. The next 68 residues were novel due to a shift in the frame of translation. Synthetic peptide antigens corresponding to amino acid residues 82 through 97 (SQLHPHAPVRVLHPSC—peptide 1; SEQ ID NO:14) and residues 61 through 74 (LPGHAPGRPWHPLC—peptide 2; SEQ ID NO:15) of the human SecR(Δexon 3,4) sequence (FIG. 2) were synthesized using a standard protocol working with Fmoc protected amino acids. Briefly, Pal resin (Advanced Chem Tech, Louisville, Ky.) was used along with coupling reagents, O-benzotriazole-N,N,N′N′-tetramethyl uronium hexafluorophosphate and 1-hydroxybenzotriazole in N′N′-diisopropyl ethyl amine. After completion of synthesis, peptides were cleaved from the resin for two hours using a trifluoroacetic acid mixture containing 82.5% (v/v) trifluoroacetic acid, 5% (w/v) phenol, 5% (v/v) distilled water, 5% (v/v) anisole, and 2.5% (v/v) triisopropylsilane. The peptides were filtered and washed with trifluoroacetic acid and dichloromethane. The combined filtrate and washes were evaporated and precipitated with ether. The peptides were dissolved in 20% acetonitrile-H₂O, lyophilized, and purified to homogeneity using semi-preparative reversed-phase HPLC. The identity of each peptide was confirmed by mass spectrometry.

In preparation for immunization of Balb/c mice, the antigenic peptides were conjugated through their carboxyl-terminal cysteine residues to keyhole limpet hemocyanin (KLH). Briefly, 3.3 mg of peptide 1 and 3.9 mg of peptide 2 were dissolved in 400 μL of 0.1 M phosphate buffer (pH 7.0) and mixed with 2 mg of maleimide-activated KLH (Pierce Biotechnology, Rockford, Ill.) dissolved in 1 mL of water. Reactions were incubated at room temperature for two hours before residual reactivity was quenched by the addition of 20 μL of 0.2 μM free cysteine. The KLH-conjugated peptides were purified by centrifugation through 10 kilodalton cut-off Centricon columns (Amicon, Millipore Corporation, Billerica, Mass.), washed repeatedly with water, and lyophilized.

Splenocytes from Balb/c mice (Jackson Laboratories, Bar Harbor, Me.) were fused to P3X63Ag8.653 myeloma cells (CRL-1580; ATCC) and the resulting hybridoma cells were clonally derived following limiting dilution into 96 well plates. Cell culture supernatants were removed from individual wells and screened for reactivity against both of the synthetic peptides, that had been conjugated to bovine serum albumin, using ELISA. Clones believed to be positive were subsequently screened by Western blot analysis using lysates derived from MiaPaCa2 cells transfected with a construct encoding an HA-tagged human SecR(Δexon 3,4) cDNA. The human SecR(Δexon 3,4) splice variant was cloned into the pBluescript KS⁺ where a hemaglutinin (HA) tag was incorporated into the Sse83871 restriction site located within exon 2. The HA-tagged hSecR variant was subcloned in the pCEP4 expression vector, sequenced, and transiently transfected into MiaPaCa2 cells using Lipofectamine 2000, according to the manufacturer's protocol (Invitrogen). Forty-eight hours after transfection, cells were harvested with cell dissociation solution (Invitrogen), washed in PBS, and resuspended in PBS lysis buffer containing 0.01% NP40 at 2×10⁷ cells per mL. An equal volume of 2× NuPage sample buffer was added to the cell lysate and heated to 70° C. for 10 minutes before being resolved on 10% glycine NuPage gels (Invitrogen).

Selected clones were subjected to additional rounds of limiting dilution to ensure their clonality, and supernatants were screened at each step following the methods described above. Milligram quantities of the 5G1 (anti-residues 82-97, peptide 1) and 8F4 (anti-residues 61-74, peptide 2) monoclonal antibodies were purified from 100 mL volumes of hybridoma culture media. One μg of each monoclonal antibody was incubated for 24 hours at 4° C. with either radioiodinated peptide 1 or peptide 2 to ensure specificity of recognition. Since these peptides lack endogenous tyrosine residues necessary for radioiodination, an aliquot of each was coupled to hydroxyphenylpropionic acid (Hpp). Each peptide was dissolved in dimethylformamide and incubated with sulfo-Hpp (Pierce) for two hours. Reactions were determined to be complete by reversed-phase C-18 HPLC analysis, and each peptide was then purified to homogeneity using semi-preparative HPLC. Radioiodination was performed using oxidative techniques with Iodo-beads (Pierce), as described elsewhere, with the product again purified to approximate specific radioactivity of 2000 Ci/mmol using reversed-phase C-18 HPLC (Dong et al., J Biol Chem., 274:903-909 (1999)). Antibody-¹²⁵I-peptide complexes were precipitated via incubation with Protein A/G-agarose beads (Pierce) and washed four times with immunoprecipitation buffer. The amount of radioactivity in each pellet was determined using a gamma counter.

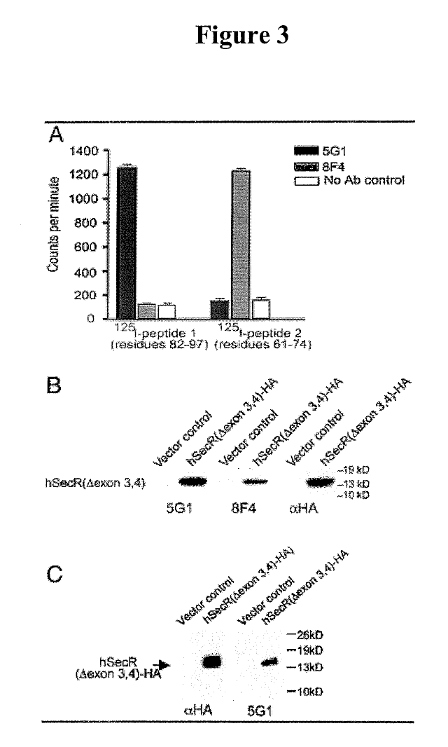

Results of the experiments described above indicated that mouse IgG antibodies were generated against two epitopes of the secretin receptor variant. The 5G1 monoclonal antibody (mAb) recognized an epitope within amino acids 82 through 97 (peptide 1), and the 8F4 mAb recognized an epitope within amino acids 61 through 74 (peptide 2), as confirmed by ELISA. Specificity was further confirmed by incubating each monoclonal antibody with radioiodinated peptides corresponding to either of these epitopes. Antibody-peptide complexes were then precipitated with protein A/G agarose beads and the radioactivity of each pellet was determined and graphed accordingly (FIG. 3A). The demonstration that the 5G1 mAb precipitated only the radioiodinated peptide 1, while the 8F4 bound solely the peptide 2, confirmed the specificity of each monoclonal antibody against the respective antigenic determinant. Wild-type secretin receptor was not recognized by either antibody.

To evaluate the ability of each mAb to recognize the full length human SecR(Δexon 3,4) polypeptide, Western blot analysis was performed. This was accomplished using lysates derived from MiaPaCa2 cells transfected with an HA-tagged version of the human SecR(Δexon 3,4) cDNA cloned into the pCEP4 expression vector. Both the 5G1 and 8F4 mAbs were able to detect a band migrating at approximate Mᵣ=14,000, consistent with the predicted mass of the variant human SecR(Δexon 3,4) polypeptide (FIG. 3B). In support of this, incubation with an anti-HA antibody detected a similar size band, while none of the antibodies detected a polypeptide of this size in lanes loaded with lysates from MiaPaCa2 cells transfected with vector only. These experiments confirmed the specificity of both the 5G1 and 8F4 antibodies, and demonstrated their ability to recognize the full length human SecR(Δexon 3,4) polypeptide.

### Example 3

### Functional Characterization of Human SecR(ΔExon 3,4)

COS cells were transfected with human SecR(Δexon 3,4) or wild-type human SecR expression constructs and studied for secretin radioligand binding and secretin-stimulated cAMP responsiveness. The human SecR(Δexon 3,4) construct in the pCEP4 expression vector was used to transfect COS cells (Lopata et al., Nucleic Acids Res., 12:5707-5717 (1984)). Seventy-two hours after transfection, the cells were used to establish the presence of secretin radioligand binding and secretin-stimulated cAMP responsiveness. Both assays were performed using techniques described elsewhere (Ulrich et al., Gastroenterology, 105:1534-1543 (1993); Ganguli et al., J Pharmacol Exper Therap., 286:593-598 (1998)). Wild type secretin receptor was used as a positive control for these studies. Strong signals for saturable secretin binding and biological activity were observed in cells transfected with the wild-type human SecR construct. Neither secretin binding nor biological activity was observed in untransfected cells or cells transfected with vector only. The signals for secretin binding and biological activity observed in cells transfected with the human SecR(Δexon 3,4) construct were similar to those in untransfected cells.

### Example 4

### Secretion of SecR(ΔExon 3,4) by Tumor Cells

The leader sequence responsible for delivery of the intact receptor to the plasma membrane was present in the human SecR(Δexon 3,4) variant, whereas the hydrophobic transmembrane segments were eliminated by the shift in coding frame and early truncation. It was therefore likely that the product was a secreted polypeptide. To determine if this was the case, MiaPaCa2 cells were transfected with the HA-tagged human SecR(Δexon 3,4) pCEP4 construct, or with the pCEP4 vector alone, and the medium was replaced after 24 hours. The cell culture supernatants were removed from adherent cells 48 hours after transfection, concentrated using a 10 kilodalton molecular weight cut-off Centricon filter, and analyzed by Western blotting. As illustrated in FIG. 3C, human SecR(Δexon 3,4) polypeptide was readily detectable in the medium by either the antibody specific for the HA-tag (control) or the antibody raised against peptide antigen 1 (mAb 5G1). By comparison, no bands were detected in the supernatant produced from cells transfected with the vector alone. These results indicate that the human SecR(Δexon 3,4) secretin receptor variant is produced and secreted in this in vitro model system, which supports the utility of the tumor-specific variant as a serum biomarker for pancreatic and potentially bile duct cancers.

### Example 5

### Detection of SecR(ΔExon 3,4) in Serum

A dual antibody-based sandwich enzyme-linked immunosorbant assay (ELISA) was designed using the two anti-human SecR(Δexon 3,4) monoclonal antibodies, 5G1 and 8F4, that recognize different epitopes within the splice variant polypeptide. To develop this assay, an appropriate polypeptide standard was needed to establish the detection limits of the ELISA protocol. The cDNA encoding the human SecR(Δexon 3,4) variant was subcloned from pBluescript KS⁻ into the pGex4T-3 bacterial expression plasmid (Amersham Biosciences, San Francisco, Calif.) for expression as a 368 amino acid fusion polypeptide with GST at the carboxy terminus. The construct was verified by automated DNA sequencing prior to transformation of BL21 E. coli. Bacterial extracts containing the GST-fusion polypeptide were loaded onto a 5 mL glutathione-Sepharose 4B resin column (Amersham Biosciences, Piscataway, N.J.). The column was washed with 15 mL of ice-cold PBS to remove unbound polypeptides, and the human SecR(Δexon 3,4)-GST fusion polypeptide was eluted with 10 mM reduced glutathione in 50 mM Tris-HCl. Glutathione was removed from the eluate by dialysis against PBS containing 0.02 mg/mL complete protease inhibitors (Roche Diagnostics, Indianapolis, Ind.). Polypeptides were quantified by measuring protein absorbance at 280 nm, analyzed on a pre-cast 4-20% gradient gel (BioRad, Hercules, Calif.), and confirmed by Western blot analysis using the 5G1 and 8F4 anti-human SecR(Δexon 3,4) mAbs and anti-GST antibodies. Serial dilutions of this polypeptide were used to optimize the ELISA protocol.

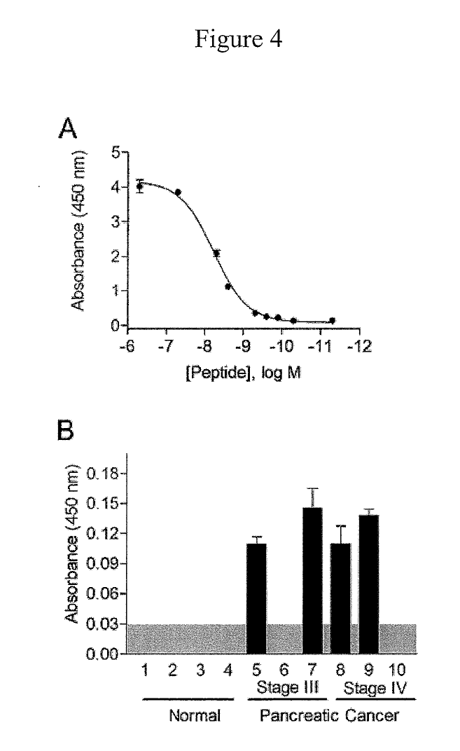

To perform the ELISA protocol, the wells of 96 well microtiter plates were coated with one μg of 8F4 (anti-residues 61-74) mAb in PBS by incubation overnight at 4° C. On the following day, the wells were washed two times with PBS and blocked with Starting Block solution (Pierce) for one hour prior to incubation overnight at 4° C. with serum from a pancreatic cancer patient, serum from a healthy human, or human SecR(Δexon 3,4)-GST polypeptide. Patient sera were analyzed blindly and diluted 1:5 or greater in Starting Block solution prior to incubation in mAb-coated microtiter plates. A set of serial dilutions of purified human SecR(Δexon 3,4)-GST polypeptide (ranging from 10⁻⁷ to 10⁻¹² M) in Starting Block solution was used as a standard for this assay. Following overnight incubation, plates were washed four times with PBS and then incubated for two hours with 100 μL per well of 1 μg/mL biotinylated 5G1 (anti-residues 82-97) mAb. After washing four times with PBS, wells were incubated with 100 μL of a 1:1000 dilution of peroxidase-conjugated mouse anti-biotin antibody (Vector Labs, Burlingame, Calif.) for two hours. Microtiter plates were again washed extensively with PBS before the addition of 3,3′,5,5′-tetramethylbenzidine (TMB)-Ultra substrate for approximately two hours, at which time 100 μL of 2M sulfuric acid were added to each well to stop the reactions. Absorbance at 450 nm was determined using a SpectraMAX 340 plate reader and SOFTmax PRO software (Molecular Devices, Sunnyvale, Calif.). The ELISA assay detected concentrations of human SecR(Δexon 3,4) polypeptide in serum that were greater than about 200 pM (FIG. 4).

The ELISA assay was used to detect the presence of human SecR(Δexon 3,4) polypeptide in ten human serum samples, six from pancreatic cancer patients and four from healthy controls. Demographic characterization of the six pancreatic cancer patients and four healthy controls are shown in Table 1. The sera were provided as blinded, coded specimens. Dilutions of the sera in blocking buffer were added to separate wells of the ELISA plates, and the human SecR(Δexon 3,4)-GST polypeptide was used as a standard control. A 1:5 dilution of the patient serum was optimal in this assay, without increasing the background signal. Higher order dilutions were also used to ensure that immunoreactivity diluted in parallel with the standard. Positive signals that were significantly above background levels were obtained from four of the six pancreatic cancer patient sera, while none of the normal sera gave detectable positive signals (Table 1 and FIG. 4). Serum levels were detected in two of three patients with stage III disease and in two of three patients with stage IV disease (Table 1). These results indicate that human SecR(Δexon 3,4) polypeptide can be detected in the serum of patients with pancreatic cancer, and that detection of SecR(Δexon 3,4) polypeptide in serum may be a valuable clinical assay.

TABLE 1
Demographics of patients having serum immunoreactive human
SecR(Δexon 3, 4) polypeptide measurements.

| Clinical status | Sex | Age | Size of tumor | | ELISA results |
|---|---|---|---|---|---|
| Healthy | F | 76 | N/A | | <200 |
| Healthy | F | 70 | N/A | | <200 |
| Healthy | F | 65 | N/A | | <200 |
| Healthy | F | 59 | N/A | | <200 |
| Pancreatic carcinoma, | F | 75 | 3 | cm | 935 ± 59* |
| stage III | | | | | |
| Pancreatic carcinoma, | M | 64 | 4.8 × 3.8 | cm | <200 |
| stage III | | | | | |
| Pancreatic carcinoma, | F | 51 | 2.8 × 1.8 | cm | 1220 ± 156* |
| stage III | | | | | |
| Pancreatic carcinoma, | M | 76 | 2.5 | cm | 939 ± 140* |
| stage IV | | | | | |
| Pancreatic carcinoma, | F | 76 | 4 | cm | 1160 ± 51* |
| stage IV | | | | | |
| Pancreatic carcinoma, | F | 52 | 3 | cm | <200 |
| stage IV | | | | | |

*p < 0.05 relative to the background activity, representing significant immunoreactivity

### Example 6

### Sensitive Detection of Human SecR(ΔExon 3,4) Polypeptides in Serum

The sensitivity of the dual antibody-based sandwich ELISA described in Example 5 was improved. In addition, the samples evaluated in Example 5 were re-evaluated and included in a larger group of samples as set forth in Table 2.

TABLE 2
Demographics of patients having serum immunoreactive human
SecR(Δexon 3, 4) polypeptide measurements.

| Clinical status | Number | Gender (M/F) | Age range (mean) |
|---|---|---|---|
| Healthy controls | 14 | 4 M/10 F | 50-79 (64) |
| Chronic pancreatitis | 10 | 7 M/3 F | 23-57 (46) |
| Pancreatic cancer | 26 | 12 M/14 F | 51-84 (66) |
| Stage 1 | 2 | 1 M/1 F | 77-84 (81) |
| Stage 2 | 6 | 4 M/2 F | 55-76 (66) |
| Stage 3 | 9 | 3 M/6 F | 51-78 (66) |
| Stage 4 | 9 | 4 M/5 F | 52-77 (64) |

The dual antibody-based sandwich ELISA was designed utilizing two anti-human SecR(Δexon 3,4) monoclonal antibodies (mAbs), 5G1 and 8F4, that recognize different epitopes within the splice variant polypeptide. Reacti-Bind Opaque 96 well microtiter plates (Pierce, Rockford, Ill.) were coated with 2 μg/mL of 8F4 (anti-residues 61-74) mAb in carbonate-bicarbonate buffer, pH 9.6 (Sigma, St. Louis, Mo.) overnight at 4° C. On the following day, the antibody solution was aspirated out, and wells were blocked for 2 hours using Starting Block solution (Pierce, Rockford, Ill.) prior to incubation with either patient sera or human SecR(Δexon 3,4)-GST for 48 hours at 4° C. Patient sera were analyzed blindly and diluted at least 1:2 in Starting Block solution prior to incubation in antibody-coated microtiter plates. A set of serial dilutions of purified human SecR(Δexon 3,4)-GST polypetpide (ranging from 10⁻⁷ to 10⁻¹² M) in Starting Block solution was used as the reference standard for this assay. Following overnight incubation, plates were washed three times with PBS-Tween and then incubated for 2 hours with 100 μL per well of 1.5 μg/mL biotinylated 5G1 (anti-residues 82-97) mAb. After washing three times with PBS-Tween, wells were incubated with 100 μL of a 1:50 dilution of Horseradish Peroxidase-Avidin D conjugate (Vector Labs, Burlingame, Calif.) for 2 hours. Microtiter plates were again washed extensively with PBS-Tween before the addition of 100 μL of SuperSignal ELISA Femto Maximum Sensitivity Substrate (Pierce, Rockford, Ill.). The human SecR spliceoform signal was then collected by measuring luminescence at 425 nm using a 2103 Envision plate reader (Perkin-Elmer, Wellesley, Mass.).

Assays were performed in a blinded manner with sera collected after informed written consent from control subjects and patients with pancreatic cancer or chronic pancreatitis (Table 2).

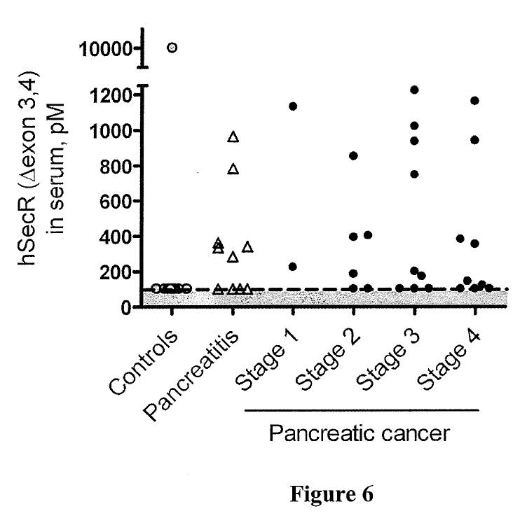

A highly sensitive and specific assay was designed to detect human SecR(Δexon 3,4) polypeptides in serum using a sandwich ELISA technique where two antibodies against independent epitopes within human SecR(Δexon 3,4) polypeptides are used in tandem. This assay was developed using the 8F4 mAb as the capture antibody to attach the human SecR(Δexon 3,4) polypeptide to the solid phase of a microwell plate and subsequently detecting its presence with a biotin-labeled 5G1 antibody. Development of this assay included the production of an appropriate polypeptide standard from which detection limits of the ELISA protocol can be established. As a result, the human SecR(Δexon 3,4) polypeptide encoding cDNA was subcloned into the pGex expression system to allow for the production and purification of a GST-tagged polypeptide from E. Coli cells. Serial dilutions of this polypeptide were used as a standard in the development of the ELISA protocol that demonstrated the ability to detect concentrations in serum greater than 100 pM human SecR(Δexon 3,4) polypeptide (FIG. 6).

The assay was subsequently used to detect the presence of human SecR(Δexon 3,4) polypeptide within sera derived from normal controls and patients with pancreatic cancer or chronic pancreatitis. Serum samples were provided as blinded, coded specimens. Dilutions of the sera in blocking buffer were applied to the ELISA protocol along with standard human SecR(Δexon 3,4)-GST control wells for each plate. Dilutions of 1:2 and 1:5 of the patient sera were used in this assay, without increasing the background signal. Higher order dilutions were also used to be certain that immunoreactivity diluted in parallel with the standard. Positive signals that were significantly above background levels were obtained from 69 percent of the pancreatic cancer patients, representing stages one through four, and in 60 percent of the chronic pancreatitis patients (FIG. 6). Only one of fourteen controls who were selected based on absence of clinical evidence of pancreatic disease or cancer had a detectable positive signal in this assay (FIG. 6). Of note, the control with the positive result had the highest level of immunoreactive variant spliceoform observed in the assay. This patient had an elevated fasting glucose and hypertension, and took multiple medications and vitamins. The demonstrated ability to detect levels of human SecR(Δexon 3,4) polypeptide in the serum of patients with pancreatic disease provides encouraging data to support the implementation of this clinical assay.

Measurable immunoreactivity of human SecR(Δexon 3,4) polypeptide was present in patients with all four stages of pancreatic cancer, including those with stage 1 disease for whom there is currently no effective serologic screening assay. It was also noteworthy that 60 percent of patients with chronic pancreatitis had similar elevations of serum levels of this biomarker. While the inability to differentiate neoplastic and inflammatory disorders of the pancreas (and possibly the biliary tree) represents a limitation in the possible ultimate clinical application of such an assay, there is still substantial need for a screening test for patients who are at risk of pancreatic cancer who have no clinical evidence of pancreatic disease. This might include patients with a strong family history of this neoplasm or those with recent diagnosis of fasting hyperglycemia. Since fasting hyperglycemia has been demonstrated in a high percentage of patients with pancreatic cancer and can be present prior to the clinical onset of disease, a strategy in which such an abnormality is followed by an assay for a sensitive serum biomarker would represent a very useful advance. The results provided herein demonstrate the importance of a sensitive and specific clinical assay for this biomarker.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for identifying a mammal as being likely to have duct cancer, said method comprising (a) determining whether a mammal has a variant SecR polypeptide, and (b) classifying said mammal as being likely to have duct cancer if said mammal has said variant SecR polypeptide, wherein said variant SecR polypeptide lacks at least a portion of the amino acid sequence set forth in SEQ ID NO:1, lacks at least a portion of the amino acid sequence set forth in SEQ ID NO:1 and SEQ ID NO:2, or comprises an amino acid sequence at least 80 percent identical to the sequence set forth in SEQ ID NO:5.

2. The method of claim 1, wherein said mammal is human.

3. The method of claim 1, wherein said duct cancer is pancreatic or bile duct cancer.

4. The method of claim 1, wherein said method comprises using blood, serum, or plasma to assess the presence or absence of said variant SecR polypeptide.

5. The method of claim 1, wherein said variant SecR polypeptide lacks at least a portion of the amino acid sequence set forth in SEQ ID NO:1.

6. The method of claim 1, wherein said variant SecR polypeptide lacks at least a portion of the amino acid sequence set forth in SEQ ID NO:1 and SEQ ID NO:2.

7. The method of claim 1, wherein said variant SecR polypeptide comprises the amino acid sequence set forth in SEQ ID NO:5.

8. The method of claim 1, wherein said method comprises using an ELISA or mass spectrometry.

9. The method of claim 1, wherein said method can detect the presence of 200 pM of said variant SecR polypeptide.

10. The method of claim 1, wherein said method can detect the presence of 100 pM of said variant SecR polypeptide.

11. An antibody preparation comprising an antibody or fragment thereof, wherein said antibody or fragment thereof binds to a polypeptide consisting of the amino acid sequence set forth in SEQ ID NO:5.

12. The antibody preparation of claim 11, wherein said antibody or fragment thereof does not bind to a polypeptide consisting of the amino acid sequence set forth in SEQ ID NO:13.

13. The antibody preparation of claim 11, wherein said antibody or fragment thereof binds to said polypeptide with an affinity greater than 10⁸ mol⁻¹ and binds, if at all, to a polypeptide consisting of the amino acid sequence set forth in SEQ ID NO:13 with an affinity less than 10⁶mol⁻¹.

14. The antibody preparation of claim 11, wherein said antibody is a monoclonal antibody.

15. The antibody preparation of claim 11, wherein said antibody or fragment thereof comprises a label selected from the group consisting of an enzyme, streptavidin, avidin, a fluorescent molecule, a luminescent molecule, a bioluminescent molecule, and a radioactive molecule.


SEQUENCE LISTING (17 sequences)

SEQ ID NO: 1 - protein, 35 aa, Homo sapien
GSLFRNCTQDGWSETFPRPNLACGVNVNDSSNEKR

SEQ ID NO: 2 - protein, 36 aa, Homo sapien
GCEGMWDNISCWPSSVPGRMVEVECPRFLRMLTSRN

SEQ ID NO: 3 - protein, 132 aa, Homo sapien
MRPHLSPPLQQLLLPVLLACAAHSTGALPRLCDVLQVLWEEQDQCLQELSREQTGDLGTEQPVPALLPAEAESHVHRGLQLLPGHAPGRPWHPLCFPEAPLHSQLHPHAPVRVLHPSCPVQLHQGRRALLLR

SEQ ID NO: 4 - protein, 111 aa, Homo sapien
AHSTGALPRLCDVLQVLWEEQDQCLQELSREQTGDLGTEQPVPALLPAEAESHVHRGLQLLPGHAPGRPWHPLCFPEAPLHSQLHPHAPVRVLHPSCPVQLHQGRRALLLR

SEQ ID NO: 5 - protein, 68 aa, Homo sapien
ALLPAEAESHVHRGLQLLPGHAPGRPWHPLCFPEAPLHSQLHPHAPVRVLHPSCPVQLHQGRRALLLR

SEQ ID NO: 6 - protein, 21 aa, Homo sapien
MRPHLSPPLQQLLLPVLLACA

SEQ ID NO: 7 - DNA, 23 bp, Homo sapien
gcagcagctactactgccggtgc

SEQ ID NO: 8 - DNA, 23 bp, Homo sapien
agccttcgcaggacctctcttgg

SEQ ID NO: 9 - DNA, 23 bp, Homo sapien
agagcaagaccagtgcctgcagg

SEQ ID NO: 10 - DNA, 22 bp, Homo sapien
agaggatgccaagggcgaccag

SEQ ID NO: 11 - DNA, 26 bp, Homo sapien
ccagctcaccatggatgatgatatcg

SEQ ID NO: 12 - DNA, 26 bp, Homo sapien
ggagttgaaggtagtttcgtggatgc

SEQ ID NO: 13 - protein, 440 aa, Homo sapien [sequence omitted]

SEQ ID NO: 14 - protein, 16 aa, Homo sapien
SQLHPHAPVRVLHPSC

SEQ ID NO: 15 - protein, 14 aa, Homo sapien
LPGHAPGRPWHPLC

SEQ ID NO: 16 - DNA, 1488 bp, Homo sapien
cggccggccggagcccgggaccctgcgcggggcgctgagctcccgagcgggcagagggcacgggcaggcggacgtcggggcgccctcggggaacgtgcgggcaccatgcgtccccacctgtcgccgccgctgcagcagctactactgccggtgctgctcgcctgcgccgcgcactcgactggagcccttccccgactatgtgacgtgctacaagtgctgtgggaagagcaagaccagtgcctgcaggaactctccagagagcagacaggagacctgggcacggagcagccagtgccagcactcctacctgctgaagctgaaagtcatgtacaccgtgggctacagctcctccctggtcatgctcctggtcgcccttggcatcctctgtgctttccggaggctccactgcactcgcaactacatccacatgcacctgttcgtgtccttcatccttcgtgccctgtccaacttcatcaaggacgccgtgctcttctcctcagatgatgtcacctactgcgatgcccacagggcgggctgcaagctggtcatggtgctgttccagtactgcatcatggccaactactcctggctgctggtggaaggcctctaccttcacacactcctcgccatctccttcttctctgaaagaaagtacctccagggatttgtggcattcggatggggttctccagccatttttgttgctttgtgggctattgccagacactttctggaagatgttgggtgctgggacatcaatgccaacgcatccatctggtggatcattcgtggtcctgtgatcctctccatcctgattaatttcatccttttcataaacattctaagaatcctgatgagaaaacttagaacccaagaaacaagaggaaatgaagtcagccattataagcgcctggccaggtccactctcctgctgatccccctctttggcatccactacatcgtcttcgccttctccccagaggacgctatggagatccagctgttttttgaactagcccttggctcattccagggactggtggtggccgtcctctactgcttcctcaatggggaggtgcagctggaggttcagaagaagtggcagcaatggcacctccgtgagttcccactgcaccccgtggcctccttcagcaacagcaccaaggccagccacttggagcagagccagggcacctgcaggaccagcatcatctgagaggctggagcagggtcacccacggacagagaccaagagaggtcctgcgaaggctgggcactgctgtgggacagccagtcttcccagcagacaccctgtgtcctccttcagctgaagatgcccctccccaggccttggactcttccgaagggatgtgaggcactgtggggcaggacaagggcctgggatttggttcgtttgctcttctgggaagagaagttcaggggtcccagaaagggacagggaaataaatggtgcctgggatgagattc

SEQ ID NO: 17 - DNA, 1703 bp, Homo sapien
acgaggccggccggagcccgggaccctgcgcggggcgctgagctcccgagcgggcagagggcacgggcaggcggacgtcggggcgccctcggggaacgtgcgggcaccatgcgtccccacctgtcgccgccgctgcagcagctactactgccggtgctgctcgcctgcgccgcgcactcgactggagcccttccccgactatgtgacgtgctacaagtgctgtgggaagagcaagaccagtgcctgcaggaactctccagagagcagacaggagacctgggcacggagcagccagtgccaggttgtgaggggatgtgggacaacataagctgctggccctcttctgtgccgggccggatggtggaggtggaatgcccgagattcctccggatgctcaccagcagaaatggttccttgttccgaaactgcacacaggatggctggtcagaaaccttccccaggcctaatctggcctgtggcgttaatgtgaacgactcttccaacgagaagcggcactcctacctgctgaagctgaaagtcatgtacaccgtgggctacagctcctccctggtcatgctcctggtcgcccttggcatcctctgtgctttccggaggctccactgcactcgcaactacatccacatgcacctgttcgtgtccttcatccttcgtgccctgtccaacttcatcaaggacgccgtgctcttctcctcagatgatgtcacctactgcgatgcccacagggcgggctgcaagctgatcatggtgctgttccagtactgcatcatggccaactactcctggctgctggtggaaggcctctaccttcacacactcctcgccatctccttcttctctgaaagaaagtacctccagggatttgtggcattcggatggggttctccagccatttttgttgctttgtgggctattgccagacactttctggaagatgttgggtgctgggacatcaatgccaacgcatccatctggtggatcattcgtggtcctgtgatcctctccatcctgattaatttcatccttttcataaacattctaagaatcctgatgagaaaacttagaacccaagaaacaagaggaaatgaagtcagccattataagcgcctggccaggtccactctcctgctgatccccctctttggcatccactacatcgtcttcgccttctccccagaggacgctatggagatccagctgttttttgaactagcccttggctcattccagggactggtggtggccgtcctctactgcttcctcaacggggaggtgcagctggaggttcagaagaagtggcagcaatggcacctccgtgagttcccactgcaccccgtggcctccttcagcaacagcaccaaggccagccacttggagcagagccagggcacctgcaggaccagcatcatctgagaggctggagcagggtcacccatggacagagaccaagagaggtcctgcgaaggctgggcactgctgtgggacagccagtcttcccagcagacaccctgtgtcctccttcagctgaagatgcccctccccaggccttggactcttccgaagggatgtgaggcactgtggggcaggacaagggcctgggatttggttcgtttgctcttctgggaagagaagttcaggggtcccagaaagggacagggaaataaatggtgcctgggatgagattc